(19) United States
(12) Patent Application Publication
(10) Pub. No.: US 2019/0209178 A1
(43) Pub. Date: Jul. 11, 2019

(54) ANEURYSM CLOSURE DEVICE

(71) Applicant: MEDINOL LTD., Tel Aviv (IL)

(72) Inventors: Jacob RICHTER, Arsuf (IL); Oleg WEIZMAN, Herzliya (IL)

(21) Appl. No.: 16/333,151

(22) PCT Filed: Sep. 5, 2017
(86) PCT No.: PCT/IB2017/001317

§ 371 Date: Mar. 13, 2019

Related U.S. Application Data
(60) Provisional application No. 62/394,564, filed on Sep. 14, 2016.

(51) Int. Cl. A61B 17/12; A61B 90/00

(52) U.S. Cl. CPC A61B 17/12113; A61B 17/12031; A61B 17/12172; A61B 2017/00867; A61B 90/39; A61B 2017/1205; A61B 17/12177

(57) ABSTRACT

The invention relates to devices, a systems, and associated methods for use, delivery, and manufacture for changing the blood flow into an aneurysm designed to induce aneurysm thrombosis and/or the exclusion from blood flow and pressure of the aneurysm in order to prevent further growth and eventual rupture. In some embodiments, the various aspects of the invention are directed to treating a cerebral aneurysm.

### CROSS-REFERENCE TO RELATED APPLICATIONS

This application claims priority to the United States provisional patent application Ser. No. 62/394,564, filed Sep. 14, 2016. Priority to the provisional patent application is expressly claimed, and the disclosure of the provisional application is hereby incorporated herein by reference in their entireties and for all purposes.

### FIELD OF THE INVENTION

The invention relates to devices, a systems, and associated methods for use, delivery, and manufacture for changing the blood flow into an aneurysm designed to induce aneurysm thrombosis and/or the exclusion from blood flow and pressure of the aneurysm in order to prevent further growth and eventual rupture.

### BACKGROUND OF THE INVENTION

A brain (cerebral) aneurysm is a protrusion of different shapes from the otherwise smooth cylindrical wall of the vessel, usually caused by a weak area in the vessel wall that gives in under blood pressure. In most cases, a brain aneurysm causes no symptoms and goes unnoticed. In some cases, the brain aneurysm ruptures, causing a hemorrhagic stroke. When a brain aneurysm ruptures in the most common area, the result is a hemorrhage (most commonly subarachnoid). Depending on the severity of the hemorrhage, permanent neurological deficiency or death may result. The most common location for brain aneurysms is in and around the network of blood vessels at the base of the brain called the circle of Willis.

Saccular aneurysm is the most common type of aneurysm. It account for 80% to 90% of all intracranial aneurysms and is the most common cause of non-traumatic subarachnoid hemorrhage (SAH). It is also known as a “berry” aneurysm because of its shape. The berry aneurysm looks like a sac or berry having a neck, or a stem and a sac (body), formed at a bifurcation or on a straight segment of an artery.

Currently, there are three primary treatments for a cerebral aneurysm: (a) craniotomy and surgical clipping, (b) endovascular coiling, and (c) flow diverters. Surgical clipping requires a craniotomy to expose the aneurysm which is then closed by attaching a clip to the neck (base) of the aneurysm, thereby providing a physical barrier to isolate the aneurismal sac. Although effective, this procedure is highly invasive and may require long recovery times. Also, it is available only for aneurisms that are close to the brain surface at an accessible position.

Endovascular coiling is a minimally-invasive procedure in which a pre-shaped coil (typically of shape-memory metal) is released into the aneurismal sac from a catheter. The coil fills the aneurismal sac causing the blood flow within the aneurismal sac to become slow and non-laminar. The blood flow disruption within the aneurismal sac results in the formation of a clot and exclusion of further blood flow into the structure, thereby preventing further expansion of the aneurysm. When successful, the thrombus eventually may be covered by a layer of endothelial cells, reforming the inner vessel wall. However, not all coiling procedures are successful. Coiling may result in aneurysm recanalization in which new routes of blood flow in the aneurism are formed, reapplying blood pressure on the aneurismal wall and further expanding it. Coiling also may require the implantation of additional devices such as stents (in order to retain the coils in the aneurism to prevent their sagging into the parent vessel) and/or the use of multiple coils (released in order to affect clotting in the aneurismal sac). The use of multiple devices increases the procedure time, treatment cost, and probability of an adverse event.

Flow diverters are stent like devices to be deployed in the parent vessel across the neck of the aneurism to alter or restrict blood flow into the aneurysm. The goal of the diverters is to cause thrombosis within the aneurismal sac. Flow diverters have limitations. For example, diverters generally should be used in relatively straight vessels and often do not perform well when the aneurysm is located at or near vessel junctions and bends. Additionally, the gaps between the struts of the diverter in many cases are too large to induce thrombosis in the aneurismal sac or may cause occlusion of the parent vessel due to clotting and/or inflammatory reactions. Finally, the diverter may cause small perforations near the aneurismal neck, causing bleeding, or may occlude nearby small diameter arteries (perforators), each of which may have neurological sequelae.

Accordingly, there is a need for an improved, efficient, and cost effective device and associated methods, for treating cerebral aneurysm, that is independent of the vessel and aneurism anatomy and that will lower the cost and duration of the procedure, as well as reduce the probability of adverse events like perforation, occlusion of nearby perforators and will have higher rate of success in excluding the aneurism.

### SUMMARY OF THE INVENTION

The present invention relates to clot-forming devices (“CFDs”), systems, and associated methods for use, delivery, and manufacture for changing the blood flow into an aneurysm designed to induce aneurysm thrombosis and/or the exclusion from blood flow and pressure of the aneurysm in order to prevent further growth and eventual rupture.

In one aspect, the invention provides a device having: (a) a central attachment member; (b) a plurality of self-expanding arms attached to the central attachment member and extending radially therefrom and (c) one or more porous panels attached to the arms and extending radially from the central attachment member; wherein the device is configured to adopt a crimped conformation having a first cross-sectional diameter and a deployed conformation having a second cross-sectional diameter that is larger than the first cross-sectional diameter, and a three-dimensional shape that is approximately spherical, semi-spherical, ovoid, or semi-ovoid. Optionally, the device may be used for aneurysm closure according to the methods described herein such that the central attachment member, arms, and mesh panels are sized such that they form a barrier or screen between a vessel and an aneurysm when the device is in the deployed conformation and positioned within the aneurysm.

In some embodiments, the device, including the central attachment member, arms, and mesh panels, are sized to fit within the lumen of a catheter when the device is in the crimped conformation. Optionally, the first cross-sectional diameter fits into a delivery system with a crossing profile less than about 10, 8, or 6 French (i.e., between about 4-10 French, 6-10 French, 4-8 French, or 6-8 French).

In some embodiments, the self-expanding arms comprise a shape memory material that has a memorized shape that defines the three-dimensional shape that is approximately spherical, semi-spherical, ovoid, or semi-ovoid.

In some embodiments, the mesh covering, formed by the one or more mesh panels, extends radially from the central attachment member to a distance of 10% or more of the length of the arms including, for example, at least 10%, 20%, 30%, 40%, 50%, 60%, 70%, 80%, 90% or more of the length of the arms, and including, for example, at least about 10%, 20%, 30%, or 40% and not more than 60%, 70%, 80%, or 90% of the length of the arms. As described herein, the mesh panel(s) may porous and may be formed from a polymer or wire mesh, a perforated polymer membrane, or a mesh of filaments.

In some embodiments, the mesh panels may contain a thrombogenic agent.

In some embodiments, the arms may be substantially linear and may further comprise straight, wavy, or spiral wires. In other embodiments, the arms define a closed shape such as an ellipse, petal shape, or reuleaux triangle. Optionally, the arms are joined by connecting struts that do not contact the attachment member. Optionally, at least one arm also contains a radio-opaque marker at or near a distal end.

In some embodiments, each arm further defines an eyelet at or near a distal end. The eyelets may be integral to the arms or attached to the arms via struts, as described herein.

The attachment member may be annular, toroidal, or any suitable shape in accordance with the principles described herein. Optionally, the attachment member contains one or more holes. Further, the device also main have a thread loop disposed through the one or more holes or eyelets and extending in the proximal direction.

In some embodiments, the device also has a guidewire disposed along a longitudinal axis of the device and through the attachment member annulus or equivalent structure. Optionally, for embodiments in which the arms further defines an eyelet at or near a distal end, the guidewire is further disposed through the eyelets when the device is in the crimped conformation. Such a configuration may be used to maintain the device in the crimped conformation either with or without the use of an external sheath.

In other embodiments, the device also may contain one or more wires attached at a first end and extending into an interior three-dimensional space defined by the arms in the deployed conformation. The wires may be attached at the first end to the attachment member or the arms.

In another aspect, the invention provides methods for closing an aneurysm and/or reducing blood flow through the neck of an aneurysm by deploying within the aneurysm any of the devices described herein. Preferably, the devices are delivered by, and deployed from a catheter.

In one embodiment, the method:
(a) provides a catheter housing a device in a crimped conformation, the device having:
   (i) a central attachment member;
   (ii) a plurality of self-expanding arms attached to the central attachment member and extending radially therefrom in a distal direction, wherein the arms define a deployed conformation having a three-dimensional shape that is approximately spherical, semi-spherical, ovoid, or semi-ovoid; and
   (iii) one or more porous panels attached to the arms and extending radially from the central attachment member;
(b) passing the catheter to a target aneurysm;
(c) inserting the device into the aneurysm;
(d) deploying the device into the deployed conformation; and
(e) withdrawing the catheter.

Optionally and as necessary, the method also includes the step of repositioning the device within the aneurysm which is performed after step (d) and repeated as desired. Repositioning may be effected through the use of a device having one or more holes in the central attachment member through which a thread loop is placed to facilitate partial or total device retrieval by the operator, as described in more detail herein.

In some embodiments, the foregoing method causes thrombosis within the aneurysm.

In another aspect, the invention provides systems having a catheter, an aneurysm closure device contained therein in a crimped conformation, and accessory structures to facilitate the delivery, positioning, and retrieval of the device. In one embodiment, the system contains:
(a) a catheter;
(b) a device in a crimped conformation within the catheter housing, the device having:
   (i) an annular central attachment member;
   (ii) a plurality of self-expanding arms attached to the central attachment member and extending radially therefrom, wherein the arms define a deployed conformation having a three-dimensional shape that is approximately spherical, semi-spherical, ovoid, or semi-ovoid; and
   (iii) one or more porous panels attached to the arms and extending radially from the central attachment member;
(c) an annular pushrod contacting a proximal side of the central attachment member; and
(d) a guidewire extending along a longitudinal axis of the catheter and disposed through a pushrod annulus, a central attachment member annulus, and a distal catheter lumen opening.

“Proximal” is a relative term that refers to the direction or side towards the entry point of the catheter into the vessel. For example, an operator withdrawing a catheter from a patient is translating the catheter in the proximal direction.

“Distal” is a relative term that refers to the direction or side away from the entry point. For example, an operator inserting a catheter into a patient is translating the catheter in a distal direction.

“Top,” when referring to a CFD, is a relative term referring to the portion of the device that is toward the aneurysmal sac or peripheral ends of the device arms.

“Bottom” when referring to a CFD, is a relative term referring to the portion of the device that is toward the aneurysmal neck or the blood vessel.

### DESCRIPTION OF DRAWINGS

FIG. 1A is a schematic diagram showing the general structure of a cerebral berry aneurism at a Y-shaped bifurcation.

FIG. 1B is a schematic diagram showing the general shape and positioning of one type of CFD deployed in the aneurism shown in FIG. 1A.

FIG. 2 illustrates exemplary but non-exhaustive arm shapes.

FIG. 3A is a plan (flat) view of a first CFD embodiment of the invention.

FIGS. 3B-C are perspective views of the first CFD embodiment shown in FIG. 3A, in possible deployed conformations.

FIG. 3D shows the first CFD embodiment shown in FIG. 3A in a crimped conformation within the lumen of a delivery sheath (e.g., a catheter).

FIG. 4A is a plan (flat) view of a second CFD embodiment of the invention.

FIGS. 4B-C are perspective views of the second CFD embodiment shown in FIG. 4A, in possible deployed conformations.

FIG. 5A is a plan (flat) view of a third CFD embodiment of the invention.

FIGS. 5B-C are perspective views of the third CFD embodiment shown in FIG. 5A, in possible deployed conformations.

FIG. 5D shows the third CFD embodiment, as shown in FIGS. 5A-C, in a crimped conformation.

FIG. 5E shows the distal end of the crimped conformation shown in FIG. 5D.

FIG. 6 shows a CFD (e.g., a CFD of the first embodiment) in a partially crimped conformation during the repositioning process.

FIGS. 7A-B are perspective views of possible deployed conformations of a fourth CFD embodiment.

FIG. 7C shows the fourth CFD embodiment, as shown in FIGS. 7A-B, in one particular crimped conformation.

FIG. 7D shows a CFD embodiment, as shown in FIGS. 7A-C, in a possible crimped conformation within the lumen of a delivery sheath (e.g., a catheter).

FIG. 8 is a perspective view of a possible deployed conformation of CFD having inner wires.

FIG. 9A is a schematic view of a CFD delivery system.

FIG. 9B is a schematic view of another CFD delivery system.

### DETAILED DESCRIPTION

The present invention provides a self-expanding Clot Forming Device (CFD) designed to be deployed within an aneurysmal sac from a catheter, and its associated delivery devices, methods for use, and methods for manufacture. The CFD may be deployed within an aneurysm located along a substantially straight portion or tortuous portion of a blood vessel wall, or an aneurysm at or near a junction or bifurcation point of a blood vessel(s). Generally, the CFD is formed from a centrally-disposed attachment member (e.g., a ring) having a plurality of arms extending therefrom in a radial pattern. The arms support a mesh covering at least the lower portion of the CFD. The CFD, when deployed, forms a three-dimensional shape that is approximately spherical, semi-spherical, ovoid, or semi-ovoid and is open at the top. The material properties and parameters allow the CFD to self-fit to the aneurismal shape.

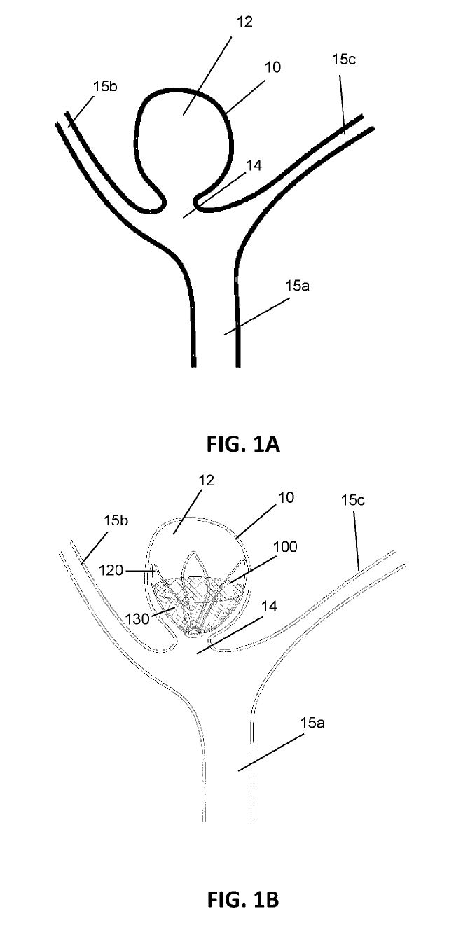

FIG. 1A schematically illustrates an aneurysm 10 having an aneurysmal sac 12 and an aneurysmal neck 14 at the junction of main blood vessel 15a and tributary branches 15b,c. FIG. 1B schematically illustrates one embodiment of a CFD 100, described in more detail below, deployed within the aneurysmal sac 12, wherein the centrally-disposed attachment member 110 is disposed within or toward the aneurysmal neck 14 with the arms 120 extending radially into the body of the aneurysmal sac 12 automatically fitting to its shape. The mesh 130 is supported by arms 120 and covers substantially all of the aneurysmal neck 14 opening. The features and specific embodiments of the CFD 100, delivery devices, methods for use, and methods for manufacture are now described in more detail.

Attachment Member

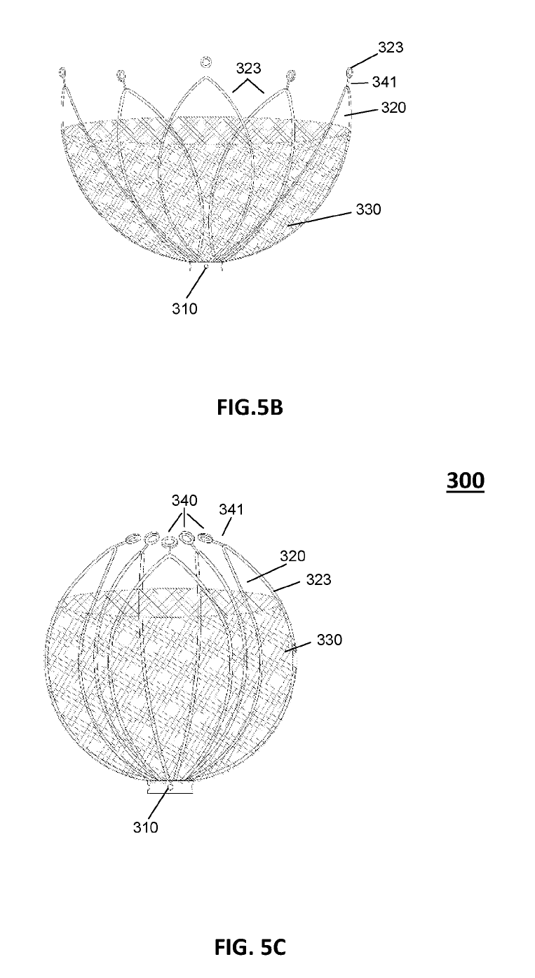

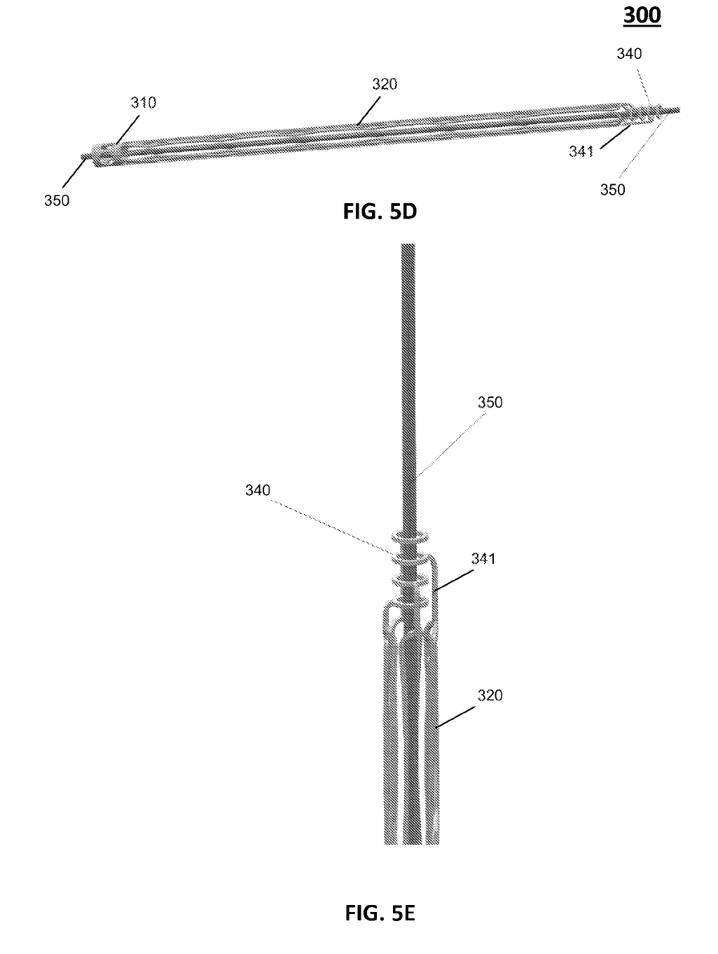

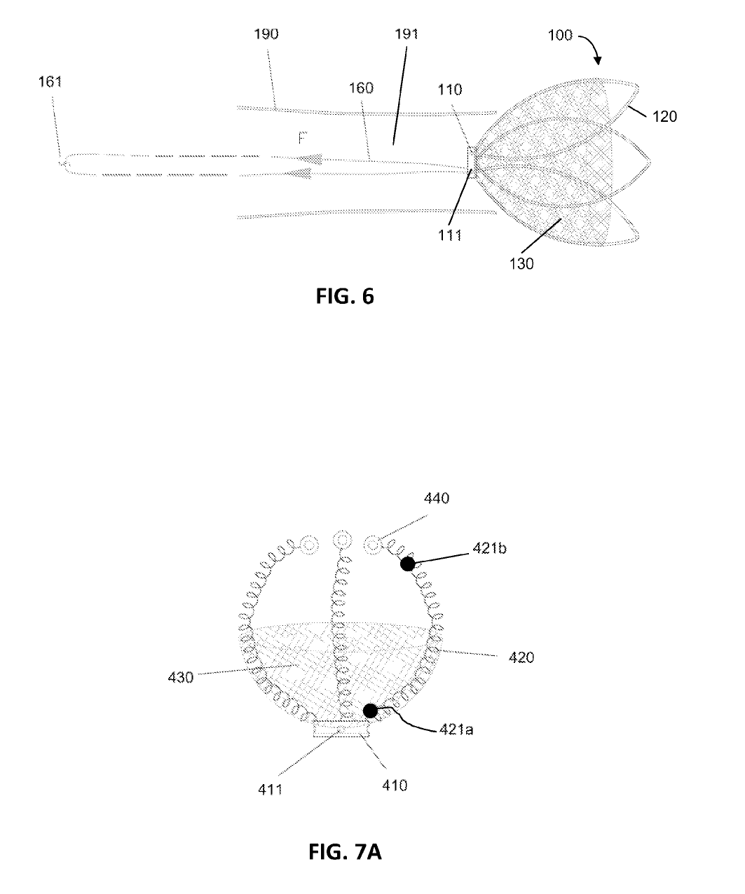

The attachment member is centrally-disposed and configured to provide an attachment point for a plurality of arms. It is sized to fit within the lumen of a catheter or inner member/jacket from which the CFD is to be deployed. There is no limitation on the shape of the attachment member, however, a generally circular shape is preferred such that the shape matches that of the deployment member lumen. In some embodiments, the attachment member is a ring (e.g., a circular ribbon), a toroid, or a disc. (See, for example, FIGS. 1B, 3B-3D, 4A-4C, 5A-5C, 7A-7B, and 8). Optionally, the attachment member is generally annular (e.g., a ring or toroid) and adapted to accommodate a centrally-disposed guide wire. (See, for example, FIGS. 5D, 7D, and 9A-B). Optionally, the attachment member has one or more holes (e.g., two, three, four, or more) adapted to accept a retrieval thread, described in more detail below. (See, for example, FIG. 6). Optionally, a radio-opaque marker is incorporated or affixed to the attachment member.

Arms

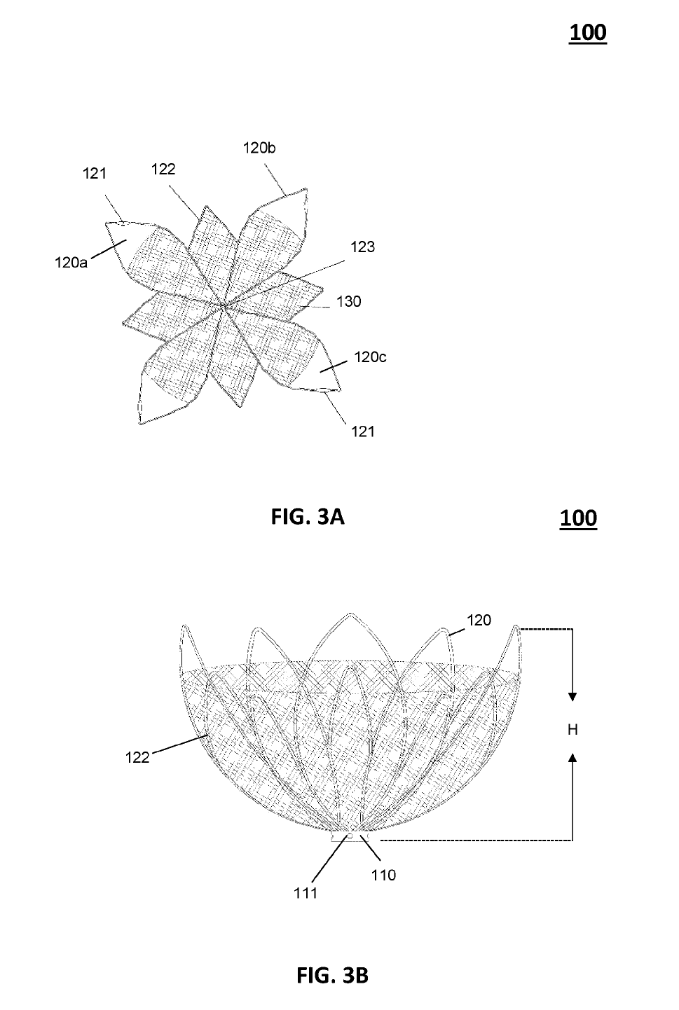

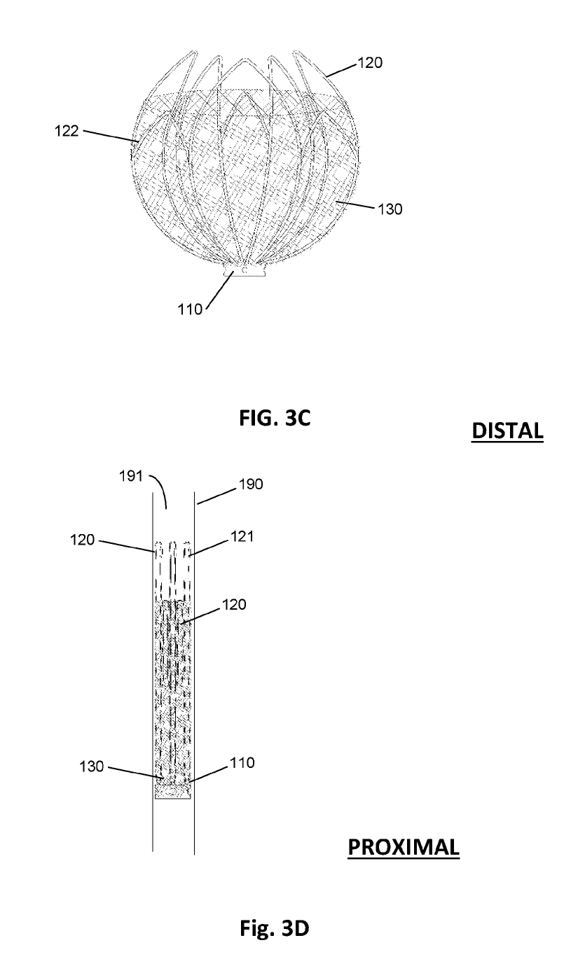

A plurality of arms (e.g., two, three, four, five, six, seven, eight, or more) are attached to the attachment member on one centrally-disposed end and extend from the attachment member in a radial pattern. The radial pattern may be symmetrical or asymmetrical, but a symmetrical pattern is preferred. The arms may be manufactured as separate elements and subsequently attached to the attachment member, or the arms and attachment member may be manufactured as a single contiguous piece. Optionally, a radio-opaque marker is incorporated or affixed to one or more of the arms. (See, for example, FIGS. 3A, 4A-4C, 5A, and 7A). Preferably, the radio-opaque marker is disposed at or near the distal end of the arm(s).

The arms are constructed to be self-expanding such that the CFD is capable of adopting a crimped conformation and a deployed conformation, the latter being its memorized shape adopted when the CFD is released from the sheath/catheter. In the crimped conformation, the distal ends of the arms are closely disposed to the longitudinal axis of the CFD such that the CFD has a first, smaller diameter adapted to be housed within the catheter or delivery device. When deployed, the arms self-expand to be disposed farther from the longitudinal axis, resulting in the CFD adopting an approximately spherical, semi-spherical, ovoid, or semi-ovoid shape, wherein the CFD has a second, larger diameter defined by the expanded arms. (compare, for example, FIGS. 3B-3C and 3D).

The arms, and optionally the attachment member, may be constructed of shape memory materials and using manufacturing methods that are well-known in the art. Specifically, the arms, and optionally the attachment member, may be constructed of known shape memory alloys including, for example, NiTi. These components can be formed by etching or laser cutting a tubing or flat sheet of material into the patterns shown. The components then may be heat treated after formation, as known by those skilled in the art, to take advantage of the shape memory characteristics and/or super elasticity. Metal surfaces may be processed chemically and/or electrochemically in order to achieve the required surface smoothness.

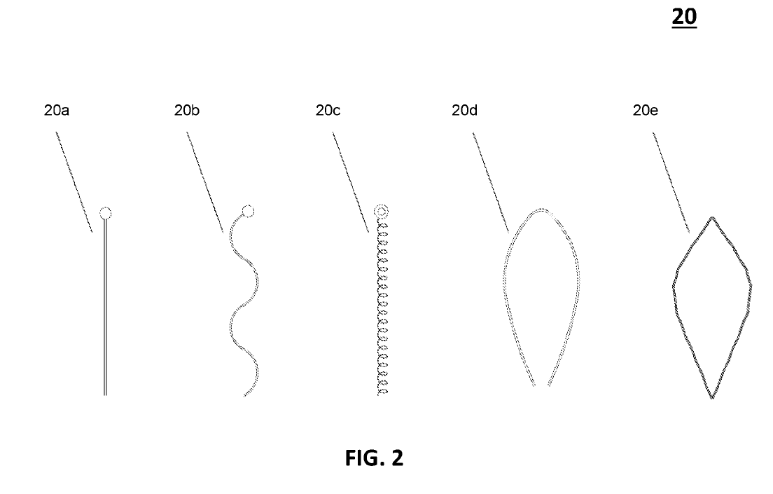

The arms may have any convenient shape suitable for supporting the mesh. For example, arms may be substantially linear elements or may define and enclose a geometric space. In the latter configuration, the geometric space is defined on its perimeter by struts and void in the interior. FIG. 2 illustrates some useful arm 20 shapes including, for example, straight 20a, wavy 20b, spiral 20c (e.g., spring or coiled), elliptical 20d, petal-shaped 20e (e.g., folium/leaf-shaped), and a reuleaux triangle (i.e., the triangular shape formed by three overlapping circles). It is understood that arms 20 in a crimped conformation are substantially linear and parallel to the longitudinal axis of the CFD. But, in the deployed conformation, arms adopt a curvilinear shape from the central to distal ends, thereby defining the spherical, semi-spherical, ovoid, or semi-ovoid shape of the CFD designed to conform to the aneurysmal sac 12. (See, for example, the deployed conformations illustrated in FIGS. 1B, 3B, 3C, 4B, 4C, 5B, 5C, 7A, 7B, and 8).

Optionally, arms further comprise eyelets at or near the distal end. The eyelets may be integral to the arms or may be attached to the arms by struts. (See, for example, FIGS. 2, 4A-4C, 5A-5C, and 7A-7B). Eyelets are configured to circumnavigate the longitudinal axis of the CFD in the crimped configuration and adapted to accept a guide wire. (See, for example, FIGS. 5D-5E).

Optionally, some or all of the arms may be attached on one or both sides to adjacent arms through connectors. Connectors are struts that attach one arm to an adjacent arm but do not attach directly to attachment member. (See, for example, FIGS. 3A-3C). Connectors may be formed from a shape memory material and, preferably, are formed from the same shape memory material as the arms. Connectors may be fabricated as separate elements and later attached to arms or may be integral and contiguous with the arms, being fabricated as a single piece. Connectors may be used to enhance CFD rigidity and/or provide additional surface area and/or support for the mesh covering.

Mesh Covering

The CFD further comprises a mesh covering supported by the arms. The mesh covering is a porous, semi-porous, or non-porous net made from a mesh of fibers or wires (e.g., metal or thermoplastic polymer such as EPTFE, polyurethane, etc.), or a perforated polymer membrane (e.g., Dacron) having holes. It is configured to limit, change, and/or reduce blood flow into the aneurysmal sac 12 when disposed across the aneurysmal neck 14. The slow and non-linear blood flow occurring through the mesh 130 is intended to cause clotting in the aneurysmal sac 12 such that the clot eventually excludes further blood flow and pressure within the sac 12, thereby preventing expansion and rupture of the aneurysm 10. In some embodiments, the mesh covering is porous to blood cells, platelets, and/or clotting factors.

The mesh is configured to restrict blood flow through the aneurysmal neck 14. Accordingly, the mesh covers at least the bottom 10%, 20%, 30%, 40%, 50%, 75% of the height of the CFD (i.e., the distance H, as illustrated in FIG. 3B, from the bottom of the attachment member to the distal end of arms 120 when CFD is in a deployed conformation). Alternatively, the mesh covers the substantially entirety of CFD. The mesh covering may be attached to the inside or the outside of the arms and/or connectors, if present. Optionally, the mesh covers an annular opening in the attachment member.

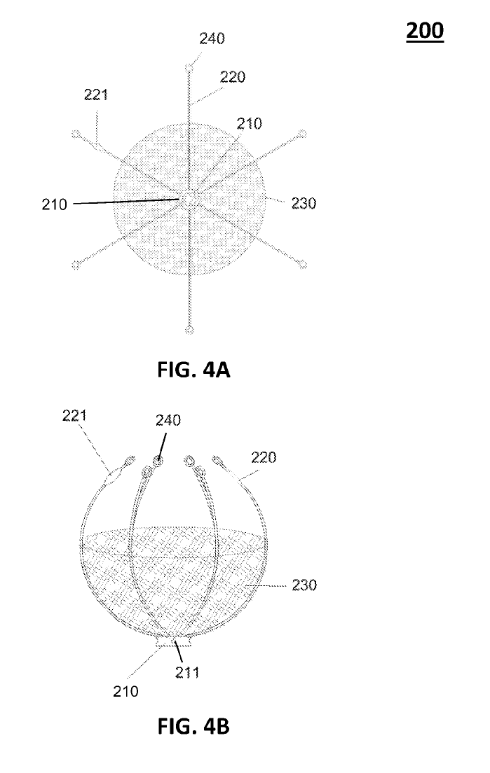

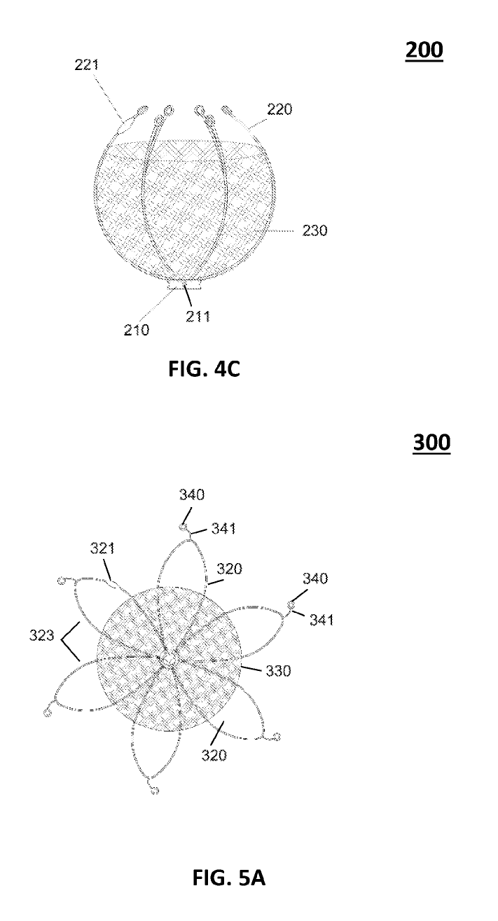

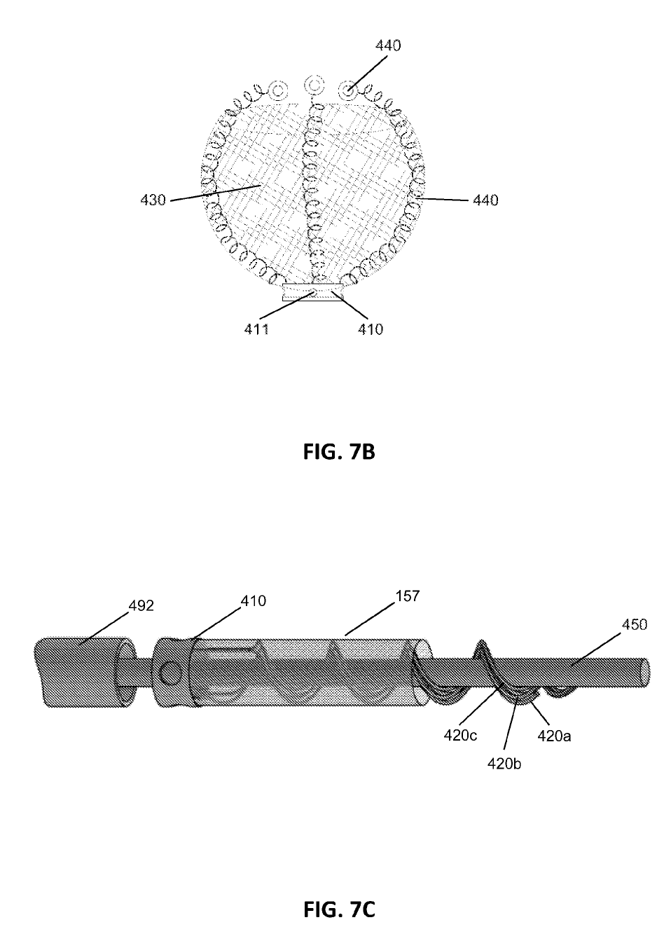

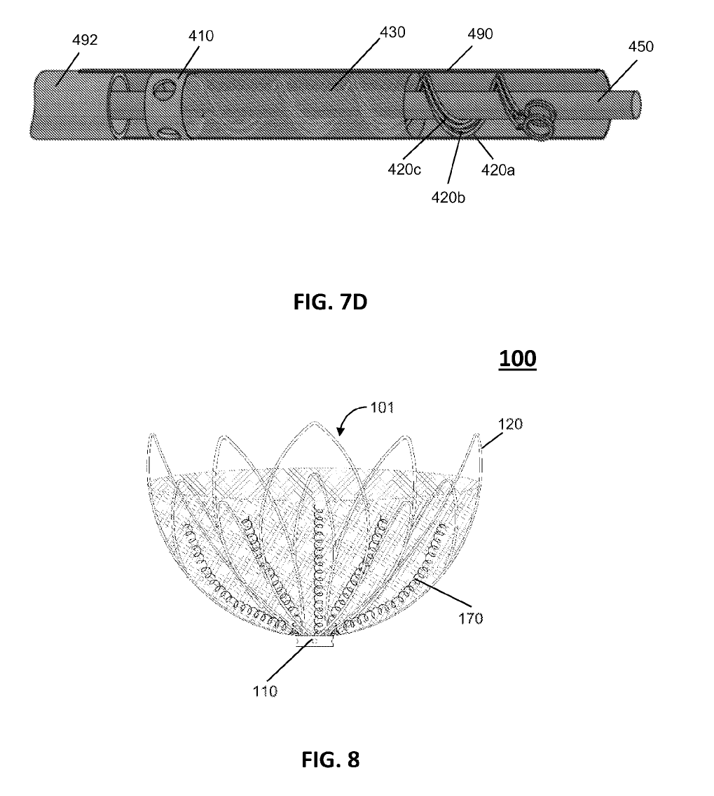

The mesh covering may be continuous (i.e., a single piece of mesh to form the covering) or discontinuous (i.e., multiple pieces of mesh that together form the covering). Continuous mesh coverings are illustrated, for example, in FIGS. 4A and 7A-7B (linear arms) and FIG. 5A (arms defining geometric shapes). In these embodiments, the mesh covering is formed by a single mesh panel. Discontinuous mesh coverings may be formed from a plurality of mesh panels. For example, the void formed by arms defining a geometric space (see, for example, arms 20d and 20e in FIG. 2, and FIG. 3A,) may be partially or totally covered by one set of mesh panels, and the void space between the arms covered by a second set of mesh panels. Mesh panels in the void space between the arms optionally may be attached to connectors, when present, for support (see, for example, connectors 122 in FIG. 3A). For embodiments in which the arms are linear, mesh panels may be attached to adjacent arms in order to form a contiguous barrier of multiple (discontinuous) mesh panels. Optionally, whether continuous or discontinuous mesh coverings are used, the mesh panel(s) may comprise one or more creases to facilitate smooth and reproducible folding when the CFD is in the crimped (folded) configuration.

For embodiments in which discontinuous mesh coverings are used, the plurality of mesh panels may be on the same side of the wire frame (i.e., arms and optional connectors) or on opposite sides of the wire frame. For example, all mesh panels may be affixed either to the outer surface or to the inner surface of the arms for support. Alternatively, some mesh panels may be affixed to the outer surface and other mesh panels may be affixed to the inner surface of the arms. In one example, the mesh panels covering or partially covering the voids formed by arms defining a geometric space may be affixed to the inside surface of the arms. Optionally, these mesh panels are creased to fold inward when crimped. And, the mesh panels covering the void space between the arms are affixed to the outside surface of the arms. Optionally, these mesh panels are creased to fold either inward or outward when crimped. In another embodiment in which the CFD is formed from linear arms, the mesh panels covering the void space between the arms may be alternated between the inside and the outside of the wire frame. For example, in a CFD having six arms and therefore defining six separate void spaces between the arms, the first, third, and fifth void space may be covered by mesh panels affixed to the inside of the wire frame, and the second, fourth, and sixth void space may be covered by mesh panels affixed to the outside of the wire frame.

The mesh covering may be configured to limit the outward deflection of the arms in the deployed conformation. For example, the self-expanding arms may be fabricated to have a resting state in which the arms define a CFD structure that is larger than desired for deployment within an aneurysm in order to ensure that the arms have a sufficient opening force to fully deploy the CFD. The circumference/diameter of the deployed conformation may be limited to a size less than the resting state of arms by appropriately limiting the size and shape of the mesh covering. The external or internal surface of CFD (i.e., the arms) may be coated with a polymer mesh by means of, for example, an electrospinning procedure, application of perforated membrane, or metal wire net.

Optionally, one or more of the mesh panels may be coated with a thrombogenic factor. Suitable thrombogenic factors include, for example, Factors VII, VIII, IX, X, XI, and XII. Thrombogenic factors may be encapsulated or incorporated into a polymer coating that is applied to the mesh panels. Alternatively, the thrombogenic factors may be affixed or adhered to the mesh panels (e.g., by dipping and drying).

CFD Construction And Design

The following implementations and embodiments are intended to illustrate additional structural and functional elements of the CFD and the principles of CFD function and design. These embodiments are not intended to be limiting. All components of the delivery catheter shall be fabricated from suitable biocompatible material for interventional invasive use.

FIG. 3A is a plan view of one embodiment of CFD 100. In this embodiment, arms 120 are formed from struts 123 defining a petal shape and are attached to a centrally-disposed attachment member 110 (shown in FIGS. 3B-D). Struts 123 are illustrated as wire which may be round. However, arms 120 may be wavy or spiral/spring-like, as described in FIG. 2. The four arms 120 are disposed in a symmetrical radial pattern resulting in a quatrefoil or flower-shaped configuration. Adjacent arms 120a, b are connected by connector 122. Like the arms 120, connectors 122 may have any configuration described in FIG. 2. Mesh 130 covers the void spaces defined by the arms 120 and connectors 122. Radio-opaque markers 121 are affixed near the distal end of arms 120a,c. This plan view may be used to represent the CFD 100 as it would be fabricated from a shape memory material prior to three-dimensional shaping.

FIG. 3B is a perspective view of CFD 100 illustrated in FIG. 3A in a deployed conformation. Attachment member 110 is illustrated as a ring having holes 111. Arms 120 extend radially from attachment member 110 and adopt a curvilinear shape over the height H. Arms 120 are interconnected by connectors 122. Mesh 130 covers the void spaces defined by the arms 120 and connectors 122 and extends more than 75% of the height. In this configuration, arms 120 define a semi-spherical or bowl-shaped form of the CFD 100.

FIG. 3C is a perspective view of CFD 100 in a different deployed conformation in which arms 120 define a substantially spherical shape open at the top.

FIG. 3D is a plan view of CFD 100 in its crimped conformation held in place within a delivery device such as a catheter 190. Attachment member 110 slideably engaged with and the same shape as the catheter lumen 191. Arms 120 are substantially straight and parallel with the central, longitudinal axis of the lumen 191. The distal and proximal ends of the catheter 190 are indicated.

FIG. 4A is a plan view of a second embodiment of CFD 200. In this embodiment, arms 220 are formed from struts extending substantially linearly from a centrally-disposed attachment member 210. Arms 220 terminate on their distal ends with integral eyelets 240. It is understood that the integral eyelets 240 illustrated in this embodiment may be substituted for the eyelet/strut configuration illustrated in the following embodiment (see, FIG. 5). Arms 220 are illustrated as straight wire which may be round. However, arms 220 may be wavy or spiral/spring-like, as described in FIG. 2. The six arms 220 are disposed in a symmetrical radial pattern, although symmetry is not required and is not a limitation of this invention. This embodiment is illustrated without connectors join adjacent arms 220, but connectors may be added, if desired. Mesh 230 is illustrated as circular and is attached to arms 220 but can be of a different shape provided that in the deployed conformation it would close the entry to the aneurism through the neck. Radio-opaque markers 221 are affixed near the distal end of at least one arm 220. This plan view may be used to represent the CFD 200 as it would be fabricated from a shape memory material prior to three-dimensional shaping.

FIG. 4B is a perspective view of CFD 200 illustrated in FIG. 4A in a deployed conformation. Attachment member 210 is illustrated as a ring having holes 211. Arms 120 extend radially from attachment member 110 and adopt a curvilinear shape over the height. Mesh 230 covers the void spaces between the arms 220 and connectors 122 and covers the lower half of the CFD 200. In this configuration, arms 120 define a substantially spherical shape open at the top.

FIG. 4C is a perspective view of CFD 200 except that mesh 230 extends more than 75% of the height of CFD 200.

FIG. 5A is a plan view of a third embodiment of CFD 300. In this embodiment, arms 320 are formed from struts 323 defining a substantially elliptical shape and are attached to a centrally-disposed attachment member 310. Struts 323 are illustrated as straight wire which may be round but, alternatively, struts 323 may have any conformation described in FIG. 2. Arms 320 have, on their distal ends, struts 341 terminating in islets 340. It is understood that this eyelet/strut configuration may be substituted for integral eyelets as described above. The six arms 320 are disposed in a symmetrical radial pattern resulting in a hexafoil or flower-shaped configuration. This embodiment is illustrated without connectors joining adjacent arms 320, but connectors may be added, if desired. Mesh 330 covers the void spaces defined by the arms 120. Radio-opaque markers 321 are affixed near the distal end of at least one arm 320. This plan view may be used to represent the CFD 300 as it would be fabricated from a shape memory material prior to three-dimensional shaping.

FIGS. 5B and 5C illustrate perspective views of alternate deployed conformations of CFD 300. FIG. 5B illustrates a CFD 300 having a semi-spherical shape and FIG. 5C illustrates a CFD 300 that is substantially spherical.

FIGS. 5D and 5E illustrate the CFD 300 in its crimped conformation. Although this illustration is presented in the context of CFD 300, it is understood that the same principles can be applied to any CFD of the invention in which eyelets are present on the distal termini of the arms. In this embodiment, CDF 300 is closed into its crimped conformation such that the plurality of eyelets 340 are aligned about the central longitudinal axis. A guide wire 350 is passed through the annulus in attachment member 310 at the proximal end of CFD 300, along the length of the longitudinal axis, and through the plurality of eyelets 340 at the distal end. The crimping pressure on the device is released and the guide wire 350 holds CFD 300 in its crimped conformation. Mesh 330 is omitted from this illustration for clarity.

In use, CFD 300 may be moved freely along guidewire 350 in its crimped conformation to facilitate accurate positioning of the device in the aneurism. CFD 300 may be ejected from a catheter and yet maintain the crimped conformation by guidewire 350. CFD 300 then may be deployed by withdrawal of guidewire 350 in the proximal direction, freeing eyelets 340, and resulting in expansion of the CFD 300 body under the self-expanding force of arms 320.

FIG. 6 illustrates an optional configuration that may be applied to any CFD embodiment described herein and which facilitates retrieval and/or repositioning of the CFD. In this embodiment, attachment member 110 further comprises one or more (e.g., two, three, four, or more) holes 111 to which a retrieval thread 160 is secured. Thread 160 may be a metal wire or polymer fiber or thread and is under the control of the operator. Preferably, thread 160 is a continuous loop that passes through hole(s) 160. After deployment from catheter 190, the operator may, on occasion, desire to retrieve or reposition CFD 100. To do so, the operator applies a pulling force (F) in the proximal direction (indicated by arrows) to partially or fully retract CFD 100 into the catheter 190 or other deployment device. The pulling force (F) causes CFD 100 to re-crimp as it comes in contact with the distal edge of the catheter lumen 191. If device retrieval is desired, the pulling force (F) is maintained until CFD 100 is retrieved entirely within the catheter lumen 191 and the catheter 190 then may be withdrawn. If repositioning is desired, it may be sufficient to only partially retrieve CFD 100 into the catheter lumen. Once repositioning is complete, pulling force (F) is released in order that CFD 100 is fully redeployed. Optionally, the repositioning process may be repeated as many times as is necessary to achieve proper CFD 100 placement within the aneurysm. Upon final CFD 100 placement, thread loop 160 may be cut at cut-point 161 by the operator and the thread 160 then may be withdrawn from the catheter, thereby freeing CFD 100 within the target aneurysm.

FIGS. 7A-7B illustrate a fourth embodiment of the invention. In this embodiment CFD 400 comprises spiral or spring-like arms 420 attached to a centrally-disposed attachment member 410, wherein each arm 420 terminates in an eyelet 440. Optionally, radio-opaque markers 421a,b are place on the attachment member 410 and one or more arms 420, respectively. Optionally, holes 411 are provided in the body of the attachment member 410. FIG. 7A is a perspective view of a substantially spherical CFD 400 in which mesh 430 covers about half of the sphere. FIG. 7B is a perspective view of a substantially spherical CFD 400 in which mesh 430 covers substantially the entire sphere.

FIG. 7C illustrates CFD 400 in one possible crimped conformation in which arms 420a,b,c are pushed into each other and a guidewire 450 is placed through the lumen of the spiral along the centrally-disposed longitudinal axis. In use, CFD 400 may slide freely over guidewire 450 to facilitate positioning while being maintained in its crimped conformation outside of the catheter lumen. For deployment, guidewire 450 is retracted in the proximal direction relative to CFD 400, thereby freeing arms 420 to adopt the deployed conformation. Optionally, CFD 400 may be held in place by push rod 492 during guide wire 450 retraction. In another embodiment of the crimped conformation (not illustrated), arms 420a,b,c are reversibly interlocked as illustrated in FIG. 7C but are not held in place by guidewire 450. Instead, arms 420a,b,c are held in the crimped conformation by virtue of their containment within a catheter lumen. Guidewire 450 terminates on the attachment member 410 and acts as a push rod to eject CFD 400 from the catheter. Upon ejection, arms 420a,b,c, automatically expand to the deployed conformation as illustrated in FIG. 7B.

FIG. 7D illustrates a related embodiment in which CFD 400 is held in its crimped conformation by guidewire 450 as above, but then loaded into an outer sheath 490 which may be a catheter or an inner tube designed to fit within a catheter.

FIG. 8 illustrates yet another optional feature that may be applied to any of the CFDs described herein. In this embodiment, the inner space 101 of the CFD 100 contains one or more metal or polymeric threads or wires 170. The wires 170 may be attached on one end to the attachment member 110 and/or arms 120. The other end of wires 170 remain free within the inner space 101. The purpose of wires 170 is to further disturb blood flow within the aneurysmal sac and accelerate thrombosis. In some embodiments, wires 170 are kinked, crimped, bent, coiled, spiraled, or spring-like.

Deployment Systems And Methods

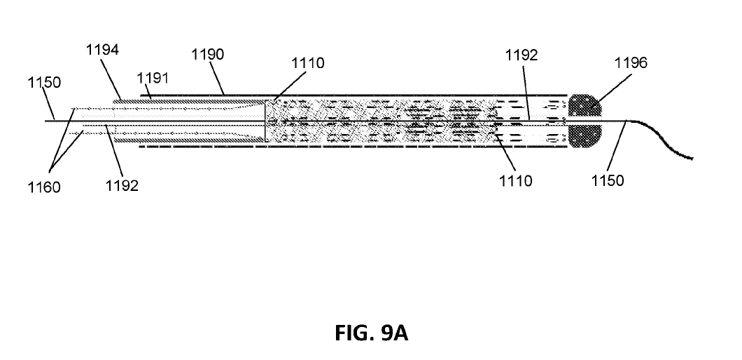

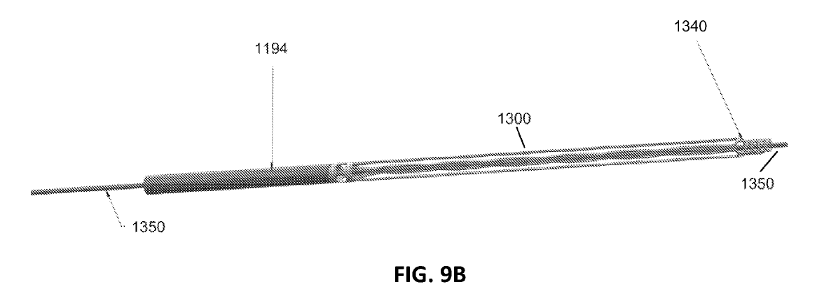

FIG. 9A illustrates one embodiment of a deployment system for a CFD including any of those described herein (e.g., CFD 100, CFD 200, CFD, 300, and CFD 400, and further including any of the optional features described herein). The deployment system comprises an outer sheath such as a catheter or other suitable external jacket 1190 having a lumen 1191, an inner member 1192 and a pushrod 1194. Lumen 1191 is adapted to house a CFD 1100 in a crimped conformation. The outer sheath 1190 may have an outer diameter in the range between 0.5 to 1.0 or between 1.0 to 3 mm. An inner member 1192 may be a flexible or a semi-flexible tube. The tube inner diameter shall be glidingly compatible with guide wire 1150. Guide wire 1150 is typically about 0.009-0.014″ or 0.018-0.035″. Distal tip 1196 is attached to the distal end of the outer sheath 1190 as described in more detail below. Distal tip 1196 provides better “deliverability” of outer sheath 1190 through the vascular system. The rounded profile (e.g., cone- or dome-shape) of distal tip 1196 facilitates smooth passage of the outer sheath 1190 through the vasculature, thereby reducing mechanical damage to the inner vessel walls (e.g., endothelial cells). Optionally, distal tip 1196 is malleable and may be formed from any suitable material including, for example, silicone-based polymers.

The delivery catheter has a pushrod 1194 inserted into the interior lumen 1191 of the outer sheath 1190 and directly abuts the CFD 1100. The pushrod 1194 has an outer diameter glidingly compatible with the inner diameter of the outer sheath 1190. The pushrod 1194 is adapted to move lengthwise inside the interior lumen 1191 of the outer sheath 1190 from the proximal end of the outer sheath 1190 to push and deploy the CFD 1100 in the target implantation site. The pushrod 1194 may be hollow in order to provide passage for an inner lumen, guide wire 1150 and threads 1160 (attached to attachment member 1110) for CFD retraction and repositioning, as described above. The outer sheath 1190 and inner tube 1192 may be equipped with radiopaque markers to be visible in X-Ray and allow a controlled positioning. In this embodiment, the CFD 1100 is held in its crimped conformation by virtue of its placement with the catheter lumen 1191. CFD 1100 deploys immediately upon ejection from the catheter lumen 1191. In one embodiment, distal tip 1196 is attached to the distal end of inner lumen 1192. After CFD 1100 deployment by outer sheath 1190 retraction, distal tip 1196 is withdrawn through the annulus of central attachment member 1110 by withdrawing inner tube 1192. In another embodiment, distal tip 1196 is attached to the distal end of outer sheath 1190 and is sufficiently malleable to allow passage of CFD 1110 through a central annulus upon deployment from outer sheath 1190.

FIG. 9B illustrates an alternate embodiment of the deployment system 1000 for use with CFDs having similar configurations to CFD 200, CFD 300, and CFD 400. The outer sheath 1190 is omitted for clarity. In this embodiment, CFD 1300 is maintained in a crimped conformation using guidewire 1350 disposed along the central longitudinal axis and through the eyelets 1340. As above, pushrod 1194 ejects CFD 1300 from the catheter lumen 1191 (not shown). Once ejected, guidewire 1350 is withdrawn in the proximal direction, freeing eyelets 1340 and causing CFD 1300 to be deployed under the opening force of the arms.

In a related embodiment, the CFD 1300 need not be housed within a catheter or other outer sheath for positioning and deployment because the guidewire 1350 without the outer sheath maintains the CFD 1300 in its crimped conformation, the pushrod 1194 and threads 1160 may be used to translocate the CFD 1300 in both the proximal and distal directions, and the pushrod 1194 may be used to hold the CFD 1300 in place while the guidewire 1350 is withdrawn for deployment. This configuration is less advantageous than catheter delivery because CFD 1300 cannot be easily retracted or partially retracted to facilitate repositioning.

Relatedly, the invention also provides methods for treating and aneurysm and/or implanting a CFD described herein. In one specific embodiment, the method comprises: (i) providing a catheter loaded with a CFD in its crimped conformation, (ii) advancing the catheter of a guidewire to a target aneurysm, (iii) placing the crimped CFD within the aneurysm, optionally based on X-ray image control, (iv) deploying the CFD, optionally based on X-ray image control, (v) repositioning the CFD, if required, and (vi) removing the catheter and the guide wire.

It will be appreciated by persons having ordinary skill in the art that many variations, additions, modifications, and other applications may be made to what has been particularly shown and described herein by way of embodiments, without departing from the spirit or scope of the invention. Therefore it is intended that scope of the invention, as defined by the claims below, includes all foreseeable variations, additions, modifications or applications.

What is claimed is:

1. A device for aneurism exclusion comprising:
(a) a central attachment member;
(b) a plurality of self-expanding arms attached to the central attachment member and extending radially therefrom and
(c) one or more panels attached to the arms and extending radially from the central attachment member, wherein the panels are porous, semi-porous, or non-porous;
wherein the device is configured to adopt a crimped conformation having a first cross-sectional diameter and a deployed conformation having a second cross-sectional diameter that is larger than the first cross-sectional diameter, and a three-dimensional shape that is approximately spherical, semi-spherical, ovoid, or semi-ovoid.

2. The device of claim 1, wherein the central attachment member, arms, and mesh panels are sized to fit within the lumen of a catheter when the device is in the crimped conformation.

3. The device of claim 1, wherein the first cross-sectional diameter fits into a delivery system with a crossing profile less than about 6 French.

4. The device of claim 1, wherein the self-expanding arms comprise a shape memory material that has a memorized shape that defines the three-dimensional shape that is approximately spherical, semi-spherical, ovoid, or semi-ovoid.

5. The device of claim 1, wherein the mesh panels extend radially from the central attachment member to a distance of 10% or more of the length of the arms.

6. The device of claim 1, wherein the central attachment member, arms, and mesh panels are sized such that they form a barrier or screen between a vessel and an aneurysm when the device is in the deployed conformation and positioned within the aneurysm.

7. The device of claim 1, wherein the arms comprise straight, wavy, or spiral wires.

8. The device of claim 1, wherein the arms define a closed shape.

9. The device of claim 8, wherein the closed shape is selected from the group consisting of approximately elliptical, approximately petal-shaped, and a reuleaux triangle.

10. The device of claim 1, wherein the arms are joined by connecting struts that do not contact the attachment member.

11. The device of claim 1, wherein at least one arm further comprises a radio-opaque marker at or near a distal end.

12. The device of claim 1, wherein each arm further comprise an eyelet at or near a distal end.

13. The device of claim 1, wherein the attachment member is annular.

14. The device of claim 13, wherein the device further comprises a guidewire disposed along a longitudinal axis of the device and through the attachment member annulus.

15. The device of claim 14, wherein the guidewire is further disposed through one or more eyelets at or near a second end of each arm when the device is in the crimped conformation.

16. The device of claim 1, wherein the attachment member further comprises one or more holes or eyelets.

17. The device of claim 16, wherein the device further comprises a thread loop disposed through the one or more holes or eyelets and extending in the proximal direction.

18. The device of claim 1, wherein the device further comprises one or more wires attached at a first end and extending into an interior space defined by the three-dimensional shape.

19. The device of claim 18, wherein the one or more wires are attached at the first end to the attachment member or one or more arms.

20. The device of claim 1, wherein the porous panels comprise a mesh or a perforated polymer membrane.

21. The device of claim 20, wherein the mesh comprises a polymer mesh a metal wire mesh, or a mesh of filaments.

22. The device of claim 1, wherein the porous panels further comprise a thrombogenic agent.

23. A method for reducing blood flow through the neck of an aneurysm, the method comprising:
(a) providing a catheter housing a device in a crimped conformation, the device comprising:
(i) a central attachment member;
(ii) a plurality of self-expanding arms attached to the central attachment member and extending radially therefrom in a distal direction, wherein the arms define a deployed conformation having a three-dimensional shape that is approximately spherical, semi-spherical, ovoid, or semi-ovoid; and
(iii) one or more panels attached to the arms and extending radially from the central attachment member, wherein the panels are porous, semi-porous, or non-porous;
(b) passing the catheter to a target aneurysm;
(c) inserting the device into the aneurysm;
(d) deploying the device into the deployed conformation; and
(e) withdrawing the catheter.

24. The method of claim 20, wherein the method further comprises, after step (d), repositioning the device within the aneurysm.

25. The method of claim 20, wherein the device causes thrombosis within the aneurysm.

26. The method of claim 20, wherein the central attachment member, arms, and mesh panels are sized such that they form a barrier or screen between a vessel and an aneurysm when the device is in the deployed conformation.

27. The method of claim 20, wherein the mesh panels extend radially from the central attachment member to a distance of 10% or more of the length of the arms.

28. A system comprising
(a) a catheter;
(b) a device in a crimped conformation within the catheter housing, the device comprising:
(i) an annular central attachment member;
(ii) a plurality of self-expanding arms attached to the central attachment member and extending radially therefrom, wherein the arms define a deployed conformation having a three-dimensional shape that is approximately spherical, semi-spherical, ovoid, or semi-ovoid; and
(iii) one or more panels attached to the arms and extending radially from the central attachment member, wherein the panels are porous, semi-porous, or non-porous;
(c) an annular pushrod contacting a proximal side of the central attachment member; and
(d) a guidewire extending along a longitudinal axis of the catheter and disposed through a pushrod annulus, a central attachment member annulus, and a distal catheter lumen opening.

29. The system of claim 28, wherein the central attachment member further comprises one or more holes and the system further comprises a thread loop disposed through the one or more holes and extending in the proximal direction through the catheter lumen.

30. The system of claim 28, wherein the mesh panels extend radially from the central attachment member to a distance of 10% or more of the length of the arms.
